(19) United States
(12) Patent Application Publication
(10) Pub. No.: US 2026/0020574 A1
(43) Pub. Date: Jan. 22, 2026

(54) USE OF AMINO ACID PEPTIDE SOLUTION PRODUCED BY DECOMPOSITION OF FISH SCALE USING BACILLUS VELEZENSIS FOR PROMOTING GERMINATION AND SEEDLING GROWTH OF GRAMINEAE

(71) Applicant: CH BIOTECH R&D CO., LTD., NANTOU COUNTY (TW)

(72) Inventors: HUI-LIANG WANG, NANTOU COUNTY (TW); YA-FANG HONG, NANTOU COUNTY (TW); CHIAO-FANG HSU, NANTOU COUNTY (TW)

(21) Appl. No.: 18/776,966

(22) Filed: Jul. 18, 2024

(51) Int. Cl. A01N 63/50; A01N 25/02; A01N 63/10; A01N 63/22; A01P 21/00; C12N 1/20; C12P 13/04; C12P 21/06; C12R 1/07

(52) U.S. Cl. CPC A01N 63/50; A01N 25/02; A01N 63/10; A01N 63/22; A01P 21/00; C12N 1/205; C12P 13/04; C12P 21/06; C12R 2001/07

(57) ABSTRACT

A use of an amino acid peptide solution produced by decomposition of fish scales using Bacillus velezensis for promoting germination and seedling growth of Gramineae is provided. The Bacillus velezensis is deposited in Leibniz Institute DSMZ-German Collection of Microorganisms and Cell Cultures GmbH with an accession number of DSM 34894. Fish scale materials are decomposed by the Bacillus velezensis to produce a fish-scale amino acid peptide solution containing amino acids and peptides. After the fish-scale amino acid peptide solution being applied to treat seeds of Gramineae, germination and seedling growth of Gramineae are effectively and specifically improved.

### BACKGROUND OF THE INVENTION

### Sequence Listing Incorporated by Reference

The Sequence Listing written in file “957-3532-SEQUENCE LISTING-JUL18-2024.xml” created on Jul. 15, 2024 with modification date Jul. 18, 2024, file size 3,449 bytes, is hereby incorporated by reference in its entirety for all purposes.

### Field of the Invention

The present invention relates to a use of an amino acid peptide solution, especially to a use of an amino acid peptide solution produced by decomposition of fish scales using Bacillus velezensis. The amino-acid peptide solution is applied to Gramineae not only for promoting germination and seedling growth, but also for improving uniformity of germination and increasing above-ground biomass.

### Description of Related Art

Fish scales are removed before cooking and eating fish and the fish scales removed are usually discarded as waste in the past. Recently, there are more and more research related to the fish scales in order to make the best use of the fish scales and reduce the waste.

The fish scales contain about 50% collagen, most of which is produced into collagen peptides by enzymatic hydrolysis, and the collagen peptides are broadly applied to skincare products and health supplements. The fish scales are rarely used as fertilizers for promotion of germination or growth of crops.

As to other fertilizers related to collagen, refer to Chinese Pat. Pub. No. 109890781A which provides an amino acid fertilizer composition prepared by treating livestock farming by-products (such as cowhide, pigskin, or cow bones) with acid, alkali, or protease to give crude collagen peptides and then hydrolyzing the crude peptides with Bacillus species. Thereby a fertilizer composed of crude collagen peptides and amino acids is obtained. Although the fertilizer composition facilitates growth of lettuces, the use of livestock farming by-products may cause zoonoses such as Bovine spongiform encephalopathy (BSE). Compared with the fertilizer from the livestock by-products, fish-based fertilizers have a lower risk of contamination and zoonoses.

Most of fish-based fertilizers available on the market are produced by composite fish materials including fish skin, fish bone, fish paste, fish powder, and recovered protein from surimi wash water. During manufacturing process, mixed strains such as yeast, lactic acid bacteria, Bacillus amyloliquefaciens, and Lactobacillus acidophilus are used. Thus, the manufacturing process is more complicated. Symbiosis Agx (American brand) is a fertilizer derived from whole freshwater fish, while Aminoterra® is derived from enzymatic hydrolysis of salmon. Compared with products obtained by simple hydrolysis of fish scales, composite fish materials may lead to inconsistent quality of fertilizer products and poor reproducibility of effect due to different sources and ratios of raw materials.

Besides, human food sources, Gramineae (Poaceae) plants, e.g., rice, maize, barley, wheat, oat, millet, and sugarcane are also main sources of animal feed. They can also be applied to various industries including architecture, pulp and paper, textile, brewing, sugar production, pharmacy, furniture, and weaving. Thus, the Gramineae have a close relationship with our lives. In the Gramineae, maize is an annual herbaceous plant and one of the important food crops. According to statistics, the worldwide production of corn in 2021 is about 1.21 billion metric tons, followed by wheat 771 million metric tons and rice 787 million metric tons. Maize shows the highest production among the food crops worldwide. Germination rate is the first key factor in higher productivity of corns. Healthy plants are vital to crop production and/or quality of crops. Corn seeds are sown directly. The poor germination rate leads to missing seedling and further results in a reduction in production per unit area and differences in vigour.

However, the poor germination rate can be resulted from poor seed quality, adverse environmental and weather conditions, etc. Seed treatment is a common way used to increase germination rate of corns because it can save seeds and pesticides applied in fields.

Based on important impact of the Gramineae on our lives and higher crop yield of the Gramineae resulted from improved germination rate, there is room for improvement and there is a need to provide a seed treatment technology for the Gramineae, which not only improves germination rate and seedling growth of the Gramineae, but also promotes uniformity of germination and increases above-ground biomass of the Gramineae. These all help users to achieve better field management and higher yield of the Gramineae.

### SUMMARY OF THE INVENTION

Therefore, it is a primary objective of the present invention to provide use of an amino acid peptide solution produced by decomposition of fish scales using Bacillus velezensis for promoting germination and seedling growth of Gramineae. The Bacillus velezensis is used to decompose the fish scales and produce the amino acid peptide solution containing peptides and amino acids. Then, the amino acid peptide solution is applied to the Gramineae for facilitating germination and seedling growth. Thus, not only germination rate and seedling growth of Gramineae are improved, uniformity of germination and above-ground biomass of Gramineae are also increased. The above improvements all help field management and improve yield of Gramineae.

In order to achieve the above objectives, a use of an amino acid peptide solution produced by decomposition of fish scales using Bacillus velezensis for promoting germination and seedling growth of Gramineae is provided. The Bacillus velezensis is deposited in Leibniz Institute DSMZ-German Collection of Microorganisms and Cell Cultures GmbH with an accession number of DSM 34894. A bacterial solution containing the Bacillus velezensis is used to decompose fish scale materials and produce the fish-scale amino acid peptide solution containing amino acids and peptides. Then, seeds of the Gramineae are premixed with the fish-scale amino acid peptide solution for seed treatment before sowing. Thereby germination rate and seedling growth of the Gramineae are effectively and specifically improved.

In some embodiments, a concentration of the amino acids and the peptides in the above fish-scale amino acid peptide solution is ranging from 25 ppm to 250 ppm.

In some embodiments, the concentration of the amino acids and the peptides in the above fish-scale amino acid peptide solution is 25 ppm.

In some embodiments, the Gramineae mentioned above includes rice, oat, wheat, barley, rye, sorghum, millet, sugarcane, maize, Job's tears, lemongrass, bamboo, water bamboo, herbage, tall fescue, bentgrass, and annual meadow grass.

In some embodiments, the Gramineae plant is maize (corn).

The present invention features on that the amino acid peptide solution produced by decomposing fish scales using Bacillus velezensis is rich in peptides and amino acids. While being applied to seeds of the Gramineae, the amino acid peptide solution not only effectively improves germination rate and shortens germination time (days) of the Gramineae seeds, but also improves uniformity of germination and increases above-ground biomass of the Gramineae. These all help improve field management and yield of the Gramineae.

### BRIEF DESCRIPTION OF THE DRAWINGS

The structure and the technical means adopted by the present invention to achieve the above and other objects can be the best understood by referring to the following detailed description of the preferred embodiments and the accompanying drawings, wherein:

FIG. 1 are photos showing observation of a colony of strain CHB200 and observation of the strain CHB200 with a microscope according to the present invention;

FIG. 2 is a figure showing analysis of contents of amino acids and peptides in decomposing products of fish scales by strain CHB200 according to the present invention;

FIG. 3 are photos showing products obtained at different stages of large-scale fermentation of fish scales by strain CHB200 according to the present invention;

FIG. 4 is a photo showing seeds of Glutinous Corn, White waxy corn from Known-You Seed Co., Ltd. with smaller size and lower germination rate according to the present invention;

FIG. 5A is a curve chart of germination rate vs. germination time of corn seeds premixed with water (control group) or a fish-scale amino acid peptide solution with a concentration of 250 ppm in the amount of 5 μL per each corn seed; there are three levels of significance flagged with stars (*p<0.05, **p<0.01, and ***p<0.001) according to the present invention;

FIG. 5B is a bar chart of mean germination time (MGT) of corn seeds premixed with water (control group) or a fish-scale amino acid peptide solution with a concentration of 250 ppm in the amount of 5 μL per each corn seed; there are three levels of significance flagged with stars (*p<0.05, **p<0.01, and ***p<0.001) according to the present invention;

FIG. 6 are photos showing side views of germination state of corn seeds on day 5 after respectively being premixed with water (control group), 25 ppm, 50 ppm, 100 ppm, and 250 ppm fish-scale amino acid peptide solution in the amount of 5 μL per each corn seed and grown under sufficient light and water according to the present invention;

FIG. 7 are photos showing top views of germination state of corn seeds on day 5 after respectively being premixed with water (control group), 25 ppm, 50 ppm, 100 ppm, and 250 ppm fish-scale amino acid peptide solution in the amount of 5 μL per each corn seed and grown under sufficient light and water according to the present invention;

FIG. 8 are photos showing side views of germination state of corn seeds on day 6 after respectively being premixed with water (control group), 25 ppm, 50 ppm, 100 ppm, and 250 ppm fish-scale amino acid peptide solution in the amount of 5 μL per each corn seed and grown under sufficient light and water according to the present invention;

FIG. 9 are photos showing top views of germination state of corn seeds on day 6 after respectively being premixed with water (control group), 25 ppm, 50 ppm, 100 ppm, and 250 ppm fish-scale amino acid peptide solution in the amount of 5 μL per each corn seed and grown under sufficient light and water according to the present invention;

FIG. 10 are bar charts of (A) fresh weight and (B) dry weight of above-ground part of corn seeds respectively being premixed with water (control group), 25 ppm, 50 ppm, 100 ppm, and 250 ppm fish-scale amino acid peptide solution in the amount of 5 μL per each corn seed and harvested on day 6; there are three levels of significance flagged with stars (*p<0.05, **p<0.01, and ***p<0.001) according to the present invention.

### DETAILED DESCRIPTION OF THE PREFERRED EMBODIMENT

A use of an amino acid peptide solution produced by using Bacillus velezensis to decompose fish scales for promoting germination and seedling growth of Gramineae according to the present invention is provided. A bacterial solution containing the Bacillus velezensis is mixed with a fish scale material to form a mixed solution. A fish-scale amino acid peptide solution is obtained after the fish scale material being decomposed by the Bacillus velezensis in the bacterial solution. The fish-scale amino acid peptide solution contains amino acids and peptides therein. When the fish-scale amino acid peptide solution is applied to Gramineae seeds, germination rate and seedling growth of Gramineae are effectively and specifically improved. Moreover, germination consistency is promoted and above-ground biomass is increased. The Bacillus velezensis is deposited in Leibniz Institute DSMZ-German Collection of Microorganisms and Cell Cultures GmbH with an accession number of DSM 34894.

16S rRNA of the present Bacillus velezensis strain has 98% similarity with 16S rRNA of type strain (standard strain) of Bacillus velezensis B268 with an accession number of CP053764.1. This standard strain is not disclosed to have fish scale decomposing ability.

The mixed solution is fermented at 30-45° C. for 12-20 hours. The mixed solution contains 7-10% (w/v) the bacterial solution containing the Bacillus velezensis, 15-40% (w/v) the fish scale material, 0.12% (w/v) buffer salts, and the rest percentage of pure water.

After steam sterilization, removal of debris by centrifugation, filtration, and concentration, a clear and concentrated fish-scale amino acid peptide solution is obtained. O-Phthaldialdehyde (OPA) test reagent is used to measure total concentration of amino acids and peptides in the fish-scale amino acid peptide solution. The result shows that total concentration of amino acids and peptides in the fish-scale amino acid peptide solution is 30,000 ppm.

In a preferred embodiment, the fish scale material includes fish scales, sodium chloride (NaCl), dipotassium phosphate (K₂HPO₄), and monopotassium phosphate (KH₂PO₄).

In a preferred embodiment, the fish scale material includes fish scales, 0.5 g/L of sodium chloride (NaCl), 0.3 g/L of dipotassium phosphate (K₂HPO₄), and 0.4 g/L of monopotassium phosphate (KH₂PO₄).

The strain CHB200 of Bacillus velezensis is obtained from waste chicken feathers. As to techniques for separating and screening strain CHB200 of Bacillus velezensis, please refer to U.S. patent application Ser. Nos. 18/760,688 and 18/772,775 filed on Jul. 1, 2024, and Jul. 15, 2024, respectively.

Moreover, as to test content of fish scale fermentation ability of the strain CHB200, please refer to U.S. patent application Ser. Nos. 18/760,688 and 18/772,775 filed on Jul. 1, 2024, and Jul. 15, 2024, respectively.

In order to learn functions and features of the present invention more completely and clearly, please refer to the following detailed descriptions and related figures. Yet the following embodiments are not intended to limit the scope of the present invention.

<Isolation and Screening of Bacillus velezensis CHB200>

Collect waste chicken feather. After washing, place 5 g of feathers into a conical flask and add 50 mL of sterile water to get a culture medium. Place the culture medium in a 37° C. incubator and culture overnight with a shaking speed at 200 rpm. Then, remove the feather from the culture medium and centrifuge the culture medium. Remove supernatant and retain precipitate after centrifugation. Next, dissolve the precipitate with 100 μL sterile water and take and spread 20 μL of re-dissolved solution evenly on Lysogeny broth (LB) solid medium. Incubate the LB solid medium in the 37° C. incubator for 24 hours.

Pick a single colony grown on the LB solid medium, inoculate the colony in 3 ml of LB broth, and culture at 37° C. with a shaking speed at 200 rpm for 24 hours to get a bacterial solution.

Measure absorbance of the bacterial solution at a wavelength of 600 nm (known as OD (optical density) 600). Inoculate the bacterial solution with OD₆₀₀ of 10 therein into 100 mL of M3 fermentation medium containing 33% fish scale powder and ferment at 37° C. for 48 hours. Then, perform hydrolysis test of fish scales. A concentration of the above bacterial solution, OD₆₀₀ of 10 is roughly 1×10⁹ CFU/mL and a composition of the M3 fermentation medium is shown in Table 1. At last, measure total concentration of amino acids in fermented solution to screen out a strain with fish scale decomposition ability.

TABLE 1

| | | concentration |
|---|---|---|
| | composition | (g/L) |
| substrate | fish scale | 330 |
| salts and buffer | NaCl | 0.5 |
| | K₂HPO₄ | 0.3 |
| | KH₂PO₄ | 0.4 |

A method of testing total concentration of amino acids is described briefly below.

Dissolve leucine in double-distilled water and prepare amino acid standard solutions with concentrations of 100-2000 ppm by serial dilution. Take 3 μL of amino acid standard solution or test sample (fermented solution), add 87 μL of ninhydrin reagent, and mix evenly to get an intermediate mixture. The ninhydrin reagent includes 0.6% ninhydrin.

The intermediate mixture is reacted at 100° C. for 10 minutes. After cooling down to room temperature naturally, add 150 μL of 95% alcohol, and mix evenly to get a final mixed solution. Measure absorbance of the final mixed solution at the wavelength of 570 nm (OD₅₇₀). Lastly, make a standard curve by absorbance of a set of the amino acid standard solutions obtained. Then, total concentration of amino acids in the test sample is calculated based on the standard curve.

Among the strains tested, a strain capable of decomposing fish scales is obtained and named as strain CHB200.

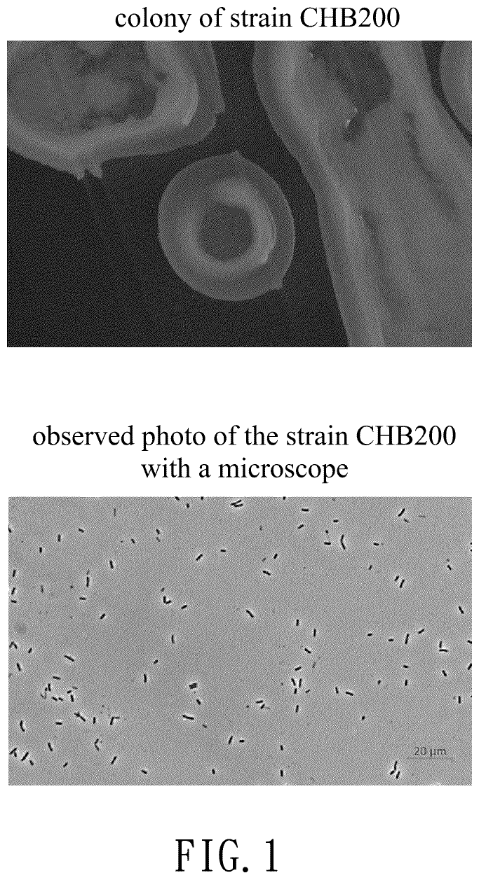

Refer to FIG. 1, a colony of the strain CHB200 is irregular round and transparent to white colored. While being observed under a microscope, the strain CHB200 is a rod with two blunt round ends. Compare 16S rRNA sequence of the strain CHB200 with sequence in Genbank database and the result shows that the strain CHB200 and the Bacillus velezensis B268 have 98% similarity in 16S rRNA sequence. 16S rRNA of the strain CHB200 is disclosed in SEQ ID NO:1 of sequence listing.

### <Test of Fish Scale Fermentation Ability of the Strain CHB200>

Inoculate the strain CHB200 into 3 ml of LB broth and culture at 37° C. with shaking at 200 rpm for 24 hours to get a bacterial solution. Then, take and inoculate the bacterial solution with OD₆₀₀ of 10, therein into 100 mL of M3 fermentation medium containing 33% fish scale powder and ferment at 37° C. to get a fermentation broth. Measure total concentration of peptides and amino acids in the fermentation broth before fermentation (0 hour) and after fermentation for 16 hrs, 24 hrs, 40 hrs, and 48 hrs. The concentration of peptides and amino acids minus the concentration of amino acids leaves the concentration of peptides.

A method of measuring total concentration of amino acids used in the test is the same as the above method using ninhydrin reagent. A method of measuring total concentration of peptides and amino acids used is using o-Phthaldialdehyde (OPA) and described briefly below.

First, prepare Leucine standard solutions. Take and add 10 μL Leucine standard solutions and test samples respectively into wells of a 96-well plate. Then, add 100 μL of OPA solution into the standard solutions and the samples and immediately measure absorbance of the standard solutions and the samples at the wavelength of 340 nm (OD₃₄₀). Next, make a standard curve by absorbance of the standard solutions obtained, and calculate total concentration of peptides and amino acids in the respective samples tested according to the standard curve. The result obtained by the OPA method minus the total concentration of amino acids obtained by the ninhydrin reagent method leaves total concentration of peptides in the sample tested.

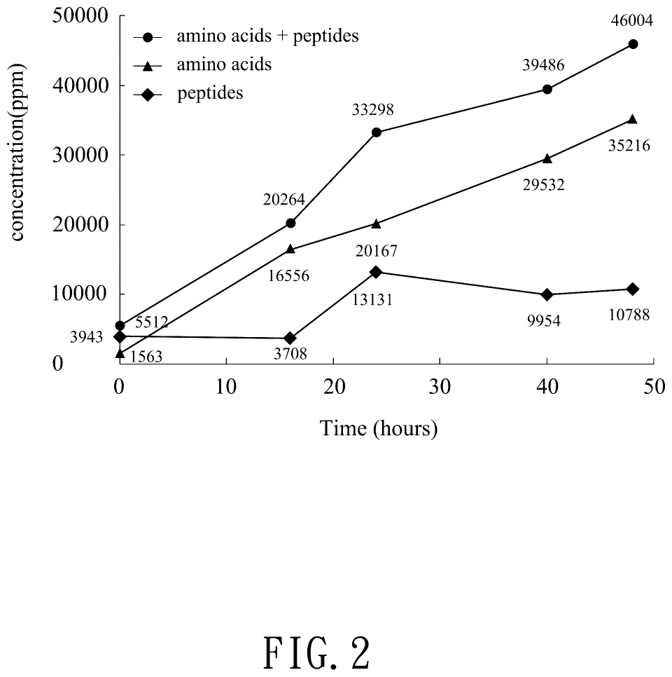

Refer to FIG. 2 and Table 2, the total concentration of amino acids and peptides in the fermentation broth is indeed increased significantly along with the increased fermentation time.

TABLE 2

| | 0 hr | 16 hrs | 24 hrs | 40 hrs | 48 hrs |
|---|---|---|---|---|---|
| total concentration of | 5512 | 20264 | 33298 | 39486 | 46004 |
| amino acids and | | | | | |
| peptides (ppm) | | | | | |
| total concentration of | 1569 | 16556 | 20167 | 29532 | 35216 |
| amino acids (ppm) | | | | | |
| total concentration of | 3943 | 3708 | 13131 | 9954 | 10788 |
| peptides (ppm) | | | | | |

### <Large Scale Fermentation of Fish Scales by the Strain CHB200>

First, activate the strain CHB200 to get a bacterial solution. Inoculate the bacterial solution in LB broth, adjust final volume to 100 mL, and incubate at 37° C. with shaking for about 16 hours to get a saturated bacterial solution. Pour the saturated bacterial solution into a 10 L fermenter and continuously incubate at 37° C. with shaking for about 2-4 hours until OD₆₀₀ of the bacterial solution reaches 4.0. Then, pour the bacterial solution with OD₆₀₀ of 4.0 into a 100-1000 L fermenter and incubate at 37° C. with shaking for about 2-4 hours until OD₆₀₀ reaches 4.0 to obtain a fermented bacterial solution.

Washed and cleaned fish scale material is placed into a 100 L-10 T fermenter, and then add buffer salts and pure water to get a M3 fermentation broth containing fish scale materials. The M3 fermentation broth is placed under 121° C. and 1.5 atm to be sterilized for 30 minutes. The sterilized M3 fermentation broth is cooled down to 30-45° C., and added with the above fermented bacterial solution to get a fermentation broth. The fermentation broth is fermented at 37° C. and stirred continuously for introduction of air. The fermentation time is 12-24 hours to obtain a primary product of fermentation. A composition of the above fermentation broth contains 7-10% (w/v) fermented bacterial solution, 15-40% (w/v) fish scale material, 0.12% (w/v) buffer salts, and the rest percentage of pure water. The preferred ratio of the fish scale material in the fermentation broth is 20-35% (w/v) and the preferred fermentation time is 16-20 hours. The composition of the M3 fermentation broth is shown in Table 1.

The primary product of fermentation is centrifuged at 3000-5000 rpm or 9000-11000 rpm by a decanter centrifuge or tubular centrifuge. Keep supernatant to obtain a debris-free fermentation broth.

Then, the debris-free fermentation broth is centrifuged at 9000-12000 rpm or 9000-11000 rpm by a disc centrifuge or a tubular centrifuge. Remove bacteria sedimentation to get a clear fermentation broth and measure total concentration of amino acids, total concentration of peptides, and nitrogen content of the clear fermentation broth.

Next, the clear fermentation broth is concentrated at 45-55° C. by a vacuum low temperature evaporator for 3-6 hours to get a concentrated fermentation broth. And measure total concentration of amino acids, total concentration of peptides, and nitrogen content of the concentrated fermentation broth.

Methods of measuring the total concentration of amino acids and peptides are as shown in the above paragraphs related to the isolation and screening of Bacillus velezensis CHB200 and the test of fish scale fermentation ability of the strain CHB200 mentioned above, while a method of measuring nitrogen content is described briefly below.

Mount a homogeneous test sample into a tin foil capsule and add silicon dioxide (SiO₂) as an adsorbent. The capsule is sealed into a cylinder or a cube. Then, use an elemental analyzer (Flash 2000, Thermo Fisher Scientific) to measure nitrogen and carbon contents in the test sample. Moreover, protein content in the test sample can be estimated by multiplying the nitrogen content by a factor of 6.25 since the average nitrogen content of proteins is found to be about 16 percent.

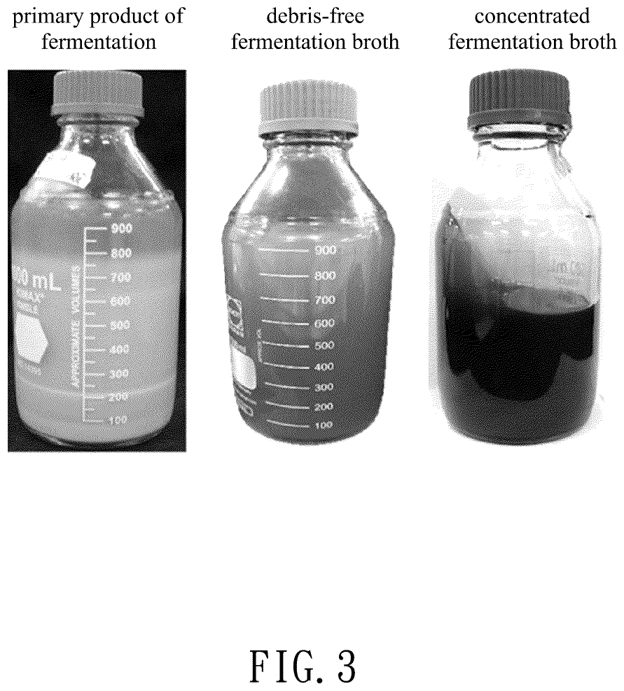

A composition analysis of the clear fermentation broth is performed by SGS Certification Taiwan, and the total concentration of peptides, amino acids, nitrogen content, and protein content in the clear fermentation broth are respectively 28164 ppm, 73080 ppm, 1.7%, and 10.56%. In the concentrated fermentation broth, the total concentration of peptides, amino acids, nitrogen content, and protein are respectively 120510 ppm, 365015 ppm, 6.9%, and 43.125%. Refer to FIG. 3, photos of the primary product of fermentation (with debris), the debris-free fermentation broth, and the concentrated fermentation broth are provided.

According to the above test results, it is learned that the strain CHB200 of the Bacillus velezensis capable of decomposing fish scales is having good fish scale decomposing ability, and able to be applied to large-scale fermentation due to the effective decomposition of fish scales within a short period of time. Thus, the strain CHB200 has potential for industrial applications. Moreover, the fermentation broth obtained from the decomposition of fish scales by the strain CHB200 is rich in amino acids and peptides, and thus able to be applied to agricultural fertilizer for promotion of seed germination and seedling growth of the Gramineae.

In a preferred embodiment, Gramineae mentioned above includes rice, oat, wheat, barley, rye, sorghum, millet, sugarcane, maize, Job's tears, lemongrass, bamboo, water bamboo, herbage, tall fescue, bentgrass, and annual meadow grass.

A fermentation solution obtained by decomposition of fish scale material by the strain CHB200 is further used for preparation of a fish-scale amino acid peptide solution which is applied to Gramineae seeds. The following are tests for showing improvement of seed germination and seedling growth of Gramineae by the fish-scale amino acid peptide solution. In the following tests, corns (maize) are test objects.

### Experiment Material and Method

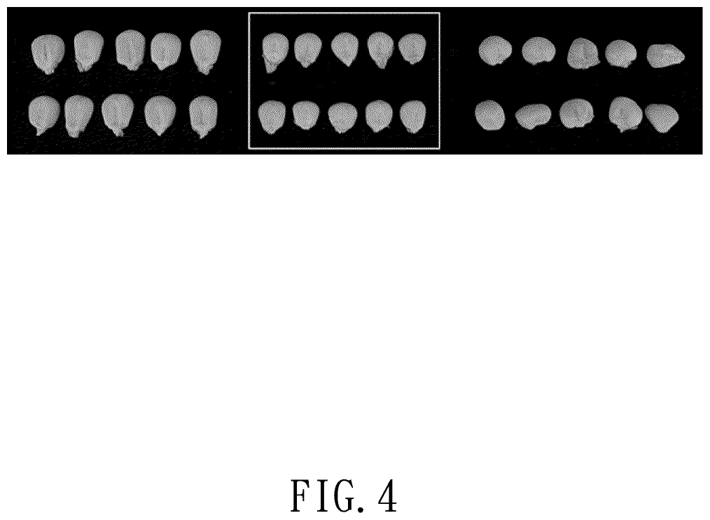

Corn seeds used are seeds of Glutinous Corn, White waxy corn from Known-You Seed Co., Ltd. with smaller size and lower germination rate (as shown in FIG. 4).

### Embodiment One

The fermentation broth obtained by decomposition of fish scales by the strain CHB200 is diluted with water to get a fish-scale amino acid peptide solution in which a total concentration of amino acids and peptides is 25 ppm.

Corn seeds are premixed with water (control group) or fish-scale amino acid peptide solution (amino acids+peptides) (treatment group) in the following manner. Put corn seeds in a 50 ml centrifuge tube and place the centrifuge tube on a shaker. Then, add water or 25 ppm fish-scale amino acid peptide solution into the centrifuge tube with shaking. The amount of water and concentration of fish-scale amino acid peptide solution required for premixing per each corn seed is 5 μL. There are three control groups and three treatment groups and the number of the corn seeds used in each of the groups is 20. Thus, the amount of water or 25 ppm fish-scale amino acid peptide solution added into the centrifuge tube per each premixing is 100 μL. After shaking and premixing, the corn seeds absorbed water or 25 ppm fish-scale amino acid peptide solution are not covered with lids for drying. Sow the corn seeds after drying.

Planting medium used is peat soil (GRAMOFLOR GmbH & Co. KG) and vermiculite (south sea vermiculite No. 2) in a ratio of 2:1. Each plug trays includes 5*4 seedling cells. Both the treatment group and the control group include 3 plugs trays and the plating medium is mounted into the respective cells. The corn seed is sown 0.5 cm below a soil surface and each cell is provided with one corn seed.

A growth temperature of the seed is 26° C. with 16 hours of daylight and 8 hours of darkness.

The mean germination time (MGT) is calculated by the following equation:

$$MGT = \frac{\sum ( fi \times di )}{N} ∘$$

wherein fi=number of seeds germinated on i^{th} day after sowing, di=number of days from the beginning of the test (after sowing), N=total number of seeds germinated at the termination of the experiment.

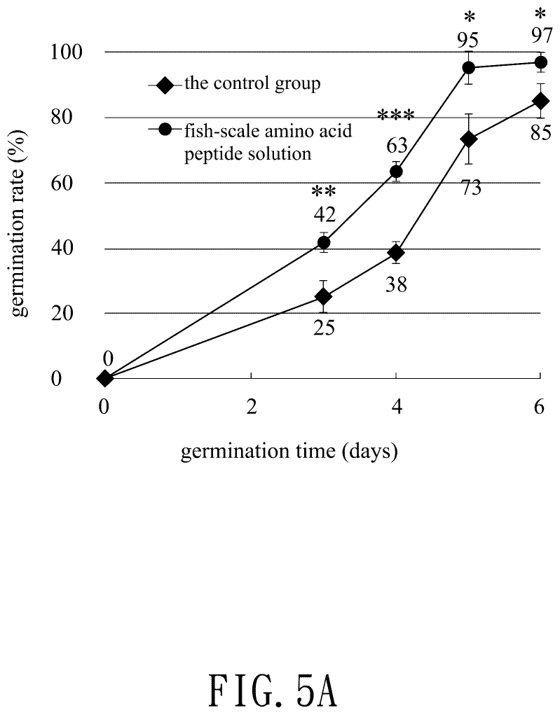

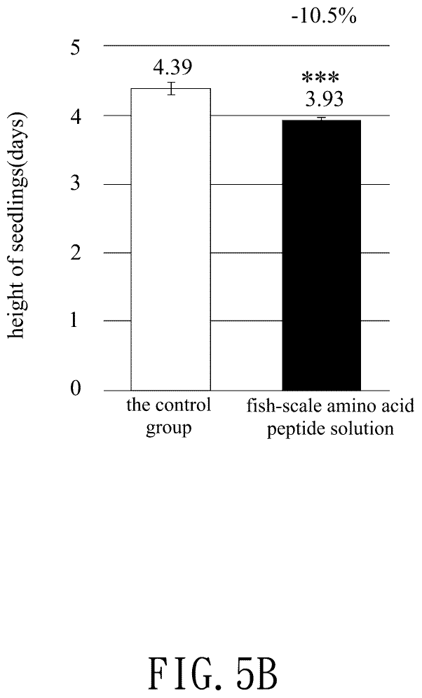

Moreover, according to a curve chart of germination rate and a bar chart of MGT shown in FIGS. 5A and 5B, it is learned that no matter the germination rate or the mean germination time (MGT), the corn seeds premixed with the fish-scale amino acid peptide solution are obviously better than the corn seeds premixed with water (control group) and there are three levels of significance (*p<0.05; **p<0.01; ***p<0.001). The germination rate is increased 12% while the MGT is shortened 10.5%.

### Embodiment Two

The fermentation broth obtained by decomposition of fish scale material by the strain CHB200 is diluted with water to get a series of fish-scale amino acid peptide solutions containing amino acids and peptides and having concentrations of 25 ppm, 50 ppm, 100 ppm, and 250 ppm.

Corn seeds are premixed with water (control group) or premixed with fish-scale amino acid peptide solution (amino acids+peptides) (treatment group) with concentrations of 25 ppm, 50 ppm, 100 ppm, and 250 ppm in the same manner as the embodiment one.

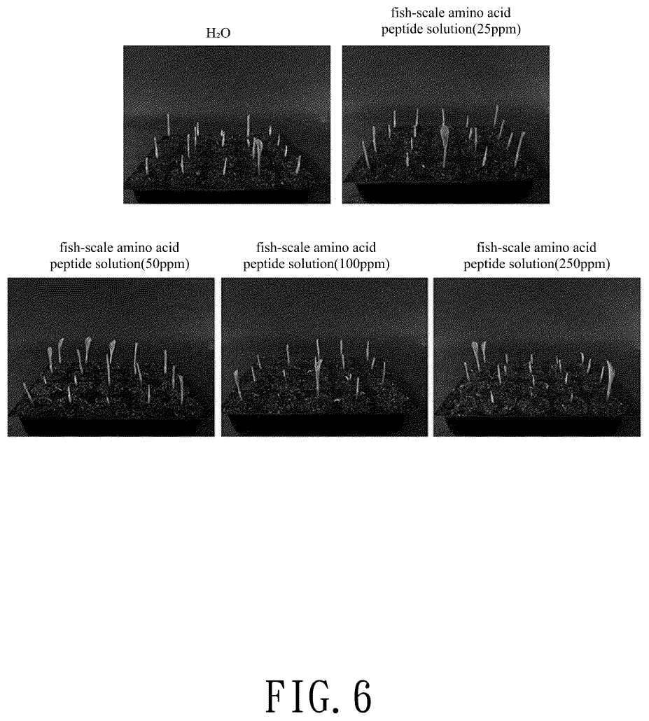

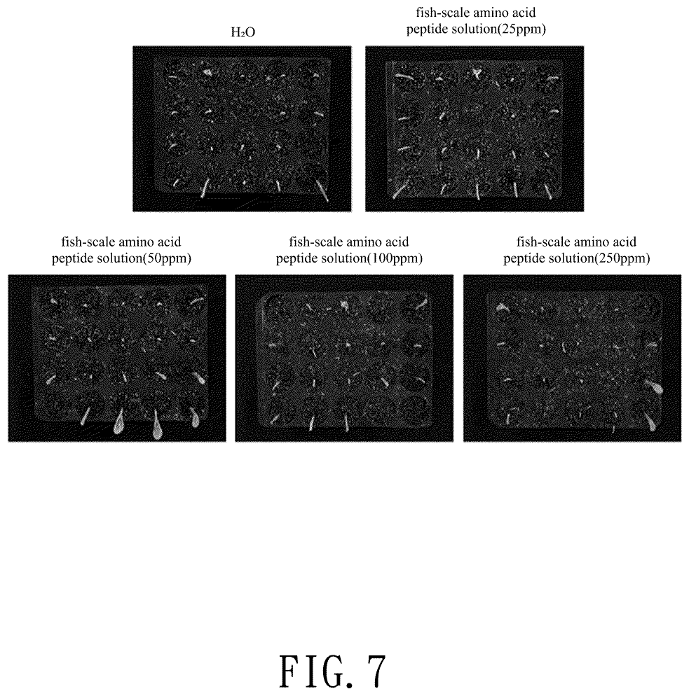

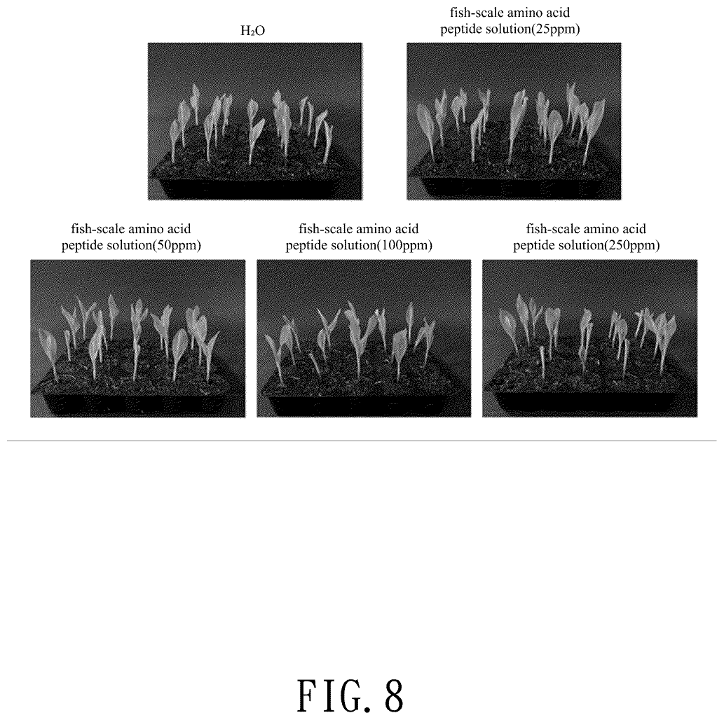

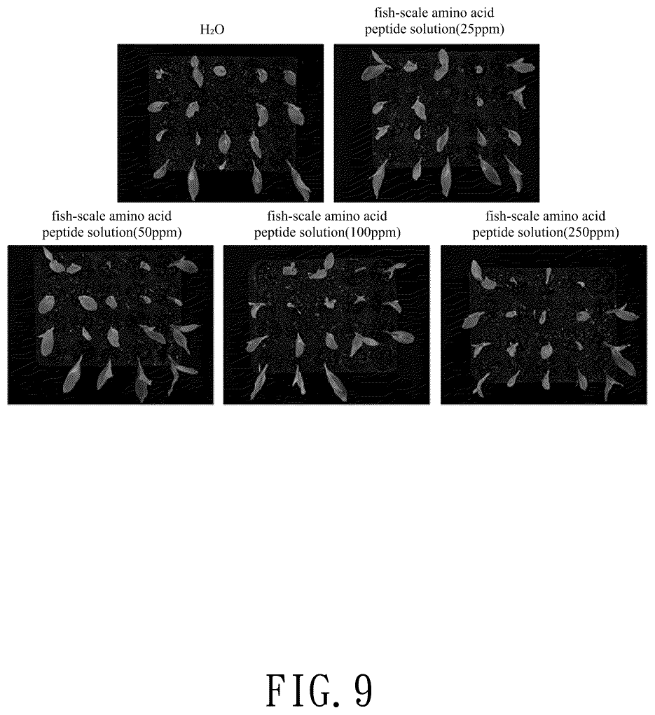

The corn seeds are provided with sufficient light and water. Then, observe and record germination state and take photos to record germination state of the corn seeds premixed with water (control group) or premixed with fish-scale amino acid peptide solutions (treatment group) having concentrations of 25 ppm, 50 ppm, 100 ppm, and 250 ppm every day. The amount of water or the respective fish-scale amino acid peptide solutions added is 5 μL per each seed. FIG. 6 and FIG. 7 are photos showing germination state of corn seeds on day 5 of the control group premixed with water or the treatment group premixed with the fish-scale amino acid peptide solutions with concentrations of 25 ppm, 50 ppm, 100 ppm, and 250 ppm in the amount of 5 μL per each seed. FIG. 8 and FIG. 9 are photos showing germination state of the corn seeds on day 6. According to photos shown in FIGS. 6-9, it is learned that under conditions with sufficient light and water, the germination rate and speed of the treatment group (premixed with the fish-scale amino acid peptide solutions with concentrations of 25 ppm, 50 ppm, 100 ppm, and 250 ppm in the amount of 5 μL per each seed) are significantly better than the control group (premixed with water in the amount of 5 μL per each seed) no matter on day 5 or day 6.

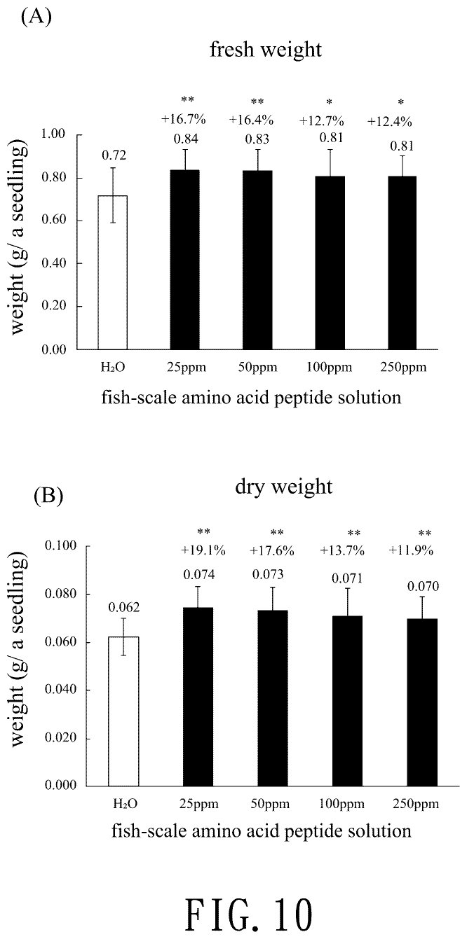

At day 6, above-ground part of the seedling is harvested after taking photos and above-ground fresh biomass is weighted by Shimadzu AP224X analytical balance. Then, the above-ground fresh biomass is placed and dried in a large type hot air circulation oven (LENON, CO-3). Use the analytical balance to weight above-ground dry biomass. The related test data is processed and analyzed with Microsoft Office Excel software tool. According to FIG. 10, it is learned that both the above-ground fresh biomass and the above-ground dry biomass of the corn seeds (the treatment group) premixed with 5 μL fish-scale amino acid peptide solutions having concentrations of 25 ppm, 50 ppm, 100 ppm, and 250 ppm per seed are clearly greater than those of the corn seeds premixed with 5 μL water/seed (control group). The optimal concentration for the treatment is 25 ppm and the above-ground fresh biomass and the above-ground dry biomass are respectively increased 16.7% and 19.1%, compared with the control group. There are three levels of significance (*p<0.05; **p<0.01; ***p<0.001). The results show that treatment of corn seeds with fish-scale amino acid peptide solutions indeed promote the growth of corn seedlings.

In summary, the amino acid peptide solution obtained by decomposition of fish scales using Bacillus velezensis according to the present invention is used to treat the corn seeds. Compared with the control group, the germination rate, the above-ground fresh biomass, and the above-ground dry biomass of the treatment group are respectively increased 12%, 16.7%, and 19.1% while the mean germination time of the treatment group is shortened 10.5%. During the tests, the amino acid peptide solutions with different concentrations applied all show no phytotoxicity. Therefore, treatment of the Gramineae seeds with the amino acid peptide solution produced by decomposition of fish scales using Bacillus velezensis can promote seed germination and seedling growth of the Gramineae and thus better field management and higher yield of the Gramineae are achieved.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A use of an amino acid peptide solution produced by decomposition of fish scales using Bacillus velezensis for seed treatment and promoting germination and seedling growth of Gramineae is provided; the Bacillus velezensis is deposited in Leibniz Institute DSMZ-German Collection of Microorganisms and Cell Cultures GmbH with an accession number of DSM 34894; a fish scale material is decomposed by a bacterial solution containing the Bacillus velezensis to produce the fish-scale amino acid peptide solution; the fish-scale amino acid peptide solution premixed with seeds of Gramineae is able to increase germination rate and seedling growth of the Gramineae effectively and specifically; the fish-scale amino acid peptide solution contains peptides and amino acids.

2. The use as claimed in claim 1, wherein a concentration of the peptides and amino acids in the fish-scale amino acid peptide solution is ranging from 25 ppm to 250 ppm.

3. The use as claimed in claim 2, wherein the concentration of the peptides and amino acids in the fish-scale amino acid peptide solution is 25 ppm, 50 ppm, 100 ppm, or 250 ppm.

4. The use as claimed in claim 3, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

5. The use as claimed in claim 4, wherein the Gramineae plant is maize.

6. The use as claimed in claim 2, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

7. The use as claimed in claim 6, wherein the Gramineae plant is maize.

8. The use as claimed in claim 1, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

9. The use as claimed in claim 8, wherein the Gramineae plant is maize.

10. A use of an amino acid peptide solution produced by decomposition of fish scales using Bacillus velezensis for promoting seedling growth of Gramineae is provided; the Bacillus velezensis is deposited in Leibniz Institute DSMZ-German Collection of Microorganisms and Cell Cultures GmbH with an accession number of DSM 34894; a fish scale material is decomposed by a bacterial solution containing the Bacillus velezensis to produce the fish-scale amino acid peptide solution; the fish-scale amino acid peptide solution premixed with seeds of Gramineae is able to increase seedling growth of the Gramineae effectively and specifically; the fish-scale amino acid peptide solution contains peptides and amino acids.

11. The use as claimed in claim 10, wherein a concentration of the peptides and amino acids in the fish-scale amino acid peptide solution is ranging from 25 ppm to 250 ppm.

12. The use as claimed in claim 11, wherein the concentration of the peptides and the amino acids in the fish-scale amino acid peptide solution is 25 ppm, 50 ppm, 100 ppm, or 250 ppm.

13. The use as claimed in claim 12, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

14. The use as claimed in claim 13, wherein the Gramineae plant is maize.

15. The use as claimed in claim 11, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

16. The use as claimed in claim 15, wherein the Gramineae plant is maize.

17. The use as claimed in claim 10, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

18. The use as claimed in claim 17, wherein the Gramineae plant is maize.


SEQUENCE LISTING (1 sequence)

SEQ ID NO: 1 - RNA, 1502 nt, Bacillus velezensis
  source 1..1502: mol_type = rRNA
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaagg